(19) United States
(12) Patent Application Publication
(10) Pub. No.: US 2024/0263113 A1
(43) Pub. Date: Aug. 8, 2024

(54) CULTURE SYSTEMS

(71) Applicant: HONDA MOTOR CO., LTD., Tokyo (JP)

(72) Inventors: Kenji Machida, Wako-shi (JP); Nozomi Shiobara, Wako-shi (JP); Shohei Kinoshita, Wako-shi (JP); Mizuho Doi, Wako-shi (JP); Minoru Goto, Wako-shi (JP); Kensuke Takahashi, Wako-shi (JP); Satoshi Shiozaki, Wako-shi (JP); Ken Harada, Wako-shi (JP); Yu Arakawa, Wako-shi (JP)

(21) Appl. No.: 18/432,104

(22) Filed: Feb. 5, 2024

(30) Foreign Application Priority Data
Feb. 8, 2023 (JP) 2023-017544

(51) Int. Cl. C12M 1/00; C12M 1/26

(52) U.S. Cl. CPC C12M 21/02; C12M 29/06; C12M 29/08; C12M 31/00; C12M 33/04

(57) ABSTRACT

A culture system includes a culture device that includes a culture tank for culturing microalgae, and a collection device that collects a culture solution. The culture tank includes a bottom portion located at the lowest position in the depth direction. The collection device includes a collection tube housed in the culture tank. The collection tube is a tubular body having an internal space. The collection tube includes a bottom side collection portion that extends along the bottom portion of the culture tank. The bottom side collection portion includes an open port formed at a site facing upward in the depth direction of the culture tank.

### CROSS-REFERENCE TO RELATED APPLICATIONS

This application is based upon and claims the benefit of priority from Japanese Patent Application No. 2023-017544 filed on Feb. 8, 2023, the contents of which are incorporated herein by reference.

### BACKGROUND OF THE INVENTION

### Field of the Invention

The present invention relates to a culture system capable of culturing microalgae and collecting the microalgae after the culturing.

### Description of the Related Art

Efforts aimed at climate change mitigation or impact reduction have continued, and toward the realization thereof, research and development in relation to the reduction of carbon dioxide emissions are being carried out. From this point of view, attention has been focused on microalgae. This is because microalgae consume carbon dioxide through photosynthesis. Therefore, a culture system for culturing microalgae is anticipated as a system that contributes to the climate change mitigation or impact reduction.

One example of the culture device is the culture device disclosed in JP 2022-157460 A. This culture device includes an accommodation portion (culture tank) made of material exhibiting flexibility, a plurality of gas supply portions, and a plurality of guide portions. The material exhibiting flexibility is, for example, vinyl bags described in JP 3213454 U.

In the case of the culture device described in JP 2022-157460 A, the plurality of gas supply portions are provided at the bottom of the accommodation portion. The guide portion is arranged directly above the gas supply portion in the accommodation portion. The gas supplied from the gas supply portion is guided by the guide portion to rise in the culture solution. As a result, convection is caused in the culture solution.

### SUMMARY OF THE INVENTION

Although not particularly shown in the drawings of JP 2022-157460 A, the culture device is provided with a collection tube for collecting the cultured microalgae and the culture solution. When gas is supplied to the culture solution from the gas supply portion with the collection tube immersed in the culture solution, there is a concern that convection is blocked by the collection tube.

An object of the present invention is to solve the above-described problems.

According to one embodiment of the present invention, there is provided a culture system including a culture device that includes a culture tank for culturing microalgae, and a collection device that collects the microalgae from the culture tank, wherein the culture tank contains a culture solution and includes a bottom portion located at a lowest position in the depth direction and an opening located at a position higher than the bottom, the culture device includes a gas supply source and a gas supply portion inserted into the culture tank through the opening, the gas supply portion includes a gas discharge port that opens in the culture solution at a lower position in the depth direction and is configured to discharge the gas supplied from the gas supply source, the collection device includes a collection tube housed in the culture tank and a suction device, the collection tube is a tubular body having an internal space, the collection tube includes a bottom side collection portion that extends along the bottom portion of the culture tank when immersed in the culture solution in the culture tank and is positioned below the gas supply portion in the depth direction, and the bottom side collection portion includes an open port formed at a site facing the opening.

In the above configuration, the gas discharged into the culture solution at the lower part of the culture tank in the depth direction rises upward in the depth direction of the culture tank based on the specific gravity difference with the culture solution. Based on this gas flow, convection occurs in the culture solution in the culture tank. That is, the microalgae and the culture solution flow. Specifically, the microalgae and the culture solution rise with the gas and then fall. This causes agitation of the microalgae and the culture solution.

Here, the bottom side collection portion, which is part of the collection tube, has an open port that opens at a site facing the opening of the culture tank. When the stream of microalgae and culture solution reaches the bottom side collection portion, the stream flows into the hollow interior of the bottom side collection portion from the open port and then returns to the culture solution from the hollow interior through the open port. By forming the open port at the bottom side collection portion in this way, the flow of microalgae and culture solution is avoided from being inhibited by the bottom side collection portion. Therefore, microalgae and culture solution are sufficiently agitated. This enhances microalgae cultivation.

The above and other objects, features, and advantages of the present invention will become more apparent from the following description when taken in conjunction with the accompanying drawings, in which a preferred embodiment of the present invention is shown by way of illustrative example.

### BRIEF DESCRIPTION OF THE DRAWINGS

FIG. 1 is a schematic system diagram of a culture system according to an embodiment of the present invention;

FIG. 2 is a cross-sectional view taken along line II-II of FIG. 1;

FIG. 3 is a perspective view showing the outline of a collection tube;

FIG. 4 is a perspective view showing the outline of a collection tube that is a shape different from the collection tube of FIG. 3;

FIG. 5 is an enlarged cross-sectional view of the vicinity of the bottom portion of the water storage tank and the culture tank;

FIG. 6 is a schematic cross-sectional view of principal parts showing a state in which the culture solution in the culture tank is being suctioned using a collection tube not having a bottom side collection portion;

FIG. 7 is a schematic cross-sectional view of principal parts showing a state in which the culture solution in the culture tank is being suctioned using a collection tube having a bottom side collection portion;

FIG. 8 is a schematic longitudinal sectional view of a culture tank constituting a culture system according to a modification example.

### DETAILED DESCRIPTION OF THE INVENTION

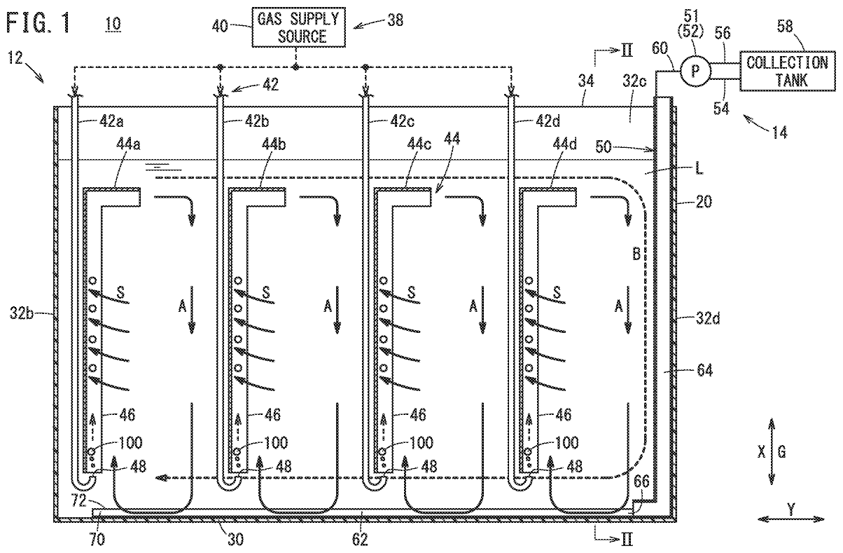

FIG. 1 is a schematic system diagram of a culture system 10 according to this embodiment. The culture system 10 includes a culture device 12 and a collection device 14.

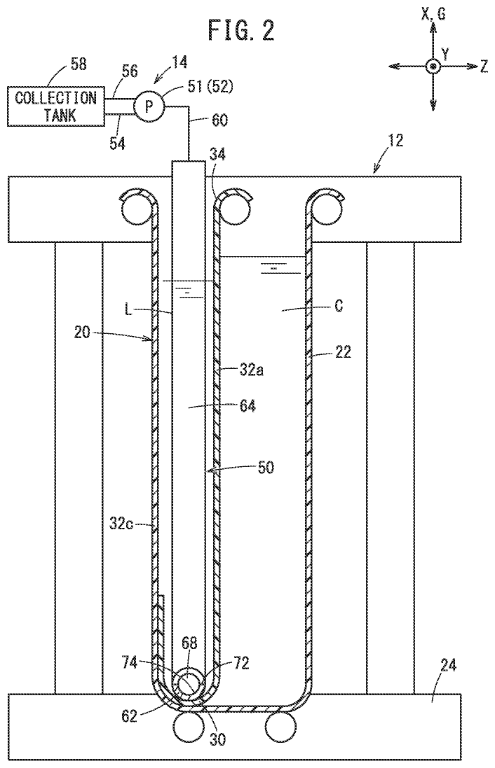

The culture device 12 will be described with reference to FIGS. 1 and 2. The culture device 12 has a culture tank 20 and a water storage tank 22. In this embodiment, the culture tank 20 and the water storage tank 22 are made of material that exhibits flexibility. A typical example of material that exhibits flexibility is linear low density polyethylene (LLDPE). The culture tank 20 and the water storage tank 22 are, for example, bag-shaped objects. The culture tank 20 and the water storage tank 22 are held by a holding frame 24 at predetermined installation points.

The material of the culture tank 20 does not have to be a material exhibiting flexibility. For example, the material of the culture tank 20 may be plastic or glass. The culture device 12 may omit the water storage tank 22.

The bag-shaped culture tank 20 has a bottom portion 30 and four side portions 32a-32d. The bottom portion 30 is located at the lowest position (lowermost portion) in the gravity direction G among all the portions of the culture tank 20. The four side portions 32a-32d are bent approximately perpendicular to the bottom portion 30 and extend in the vertical direction. The top of the culture tank 20 is an opening 34. As can be seen, the opening 34 is located higher than the bottom portion 30. The culture tank 20 is formed in a substantially quadrangular tubular shape by the bottom portion 30, the four side portions 32a-32d, and the opening 34.

The direction of extension along the height of the four side portions 32a-32d is defined as the depth direction X of the culture tank 20. In this aspect, the depth direction X of the culture tank 20 approximately matches the gravity direction G. Therefore, in this aspect, an inclination angle of the culture tank 20 in the depth direction X with respect to the gravity direction G is approximately zero. That is, the culture tank 20 is in an upright position.

As can be understood by comparing FIGS. 1 and 2 with each other, the length of side portions 32a and 32c along the horizontal direction is greater than the length of side portions 32b and 32d along the horizontal direction. Hereinafter, the horizontal direction in which the side portions 32a and 32c extend is referred to as the extension direction Y (see FIG. 1) of the culture tank 20. The side portions 32b, 32d are located at the end portions of the culture tank 20 in the extension direction Y. The horizontal direction in which the side portions 32b and 32d extend is referred to as the width direction Z (see FIG. 2) of the culture tank 20.

The interior of the culture tank 20 is a space for accommodating microalgae and the culture solution L. As specific examples of the microalgae, there may be cited the “Honda DREAMO strain” deposited at the National Institute for Product Evaluation Technology Patent Microorganism Depositary Center. In this instance, the National Institute for Product Evaluation Technology Patent Microorganism Depositary Center is located in Room 122, 2-5-8 Kazusa Kamatari, Kisarazu City, Chiba Prefecture. The date of deposit of the Honda DREAMO strain is Apr. 22, 2016, and the accession number is FERM BP-22306. The culture solution L is typically water.

The culture device 12 has a gas supply device 38. As shown in FIG. 1, in this aspect, the gas supply device 38 has a gas supply source 40, a gas supply portion 42, and a guide portion 44. The gas supply source 40 is, for example, a tank storing a gas containing carbon dioxide. The gas containing carbon dioxide is, for example, an exhaust gas of a factory. In this aspect, the gas supply portion 42 has a first gas supply portion 42a to a fourth gas supply portion 42d, and the guide portion 44 has a first guide portion 44a to a fourth guide portion 44d.

The first to fourth gas supply portions 42a to 42d and the first to fourth guide portions 44a to 44d are inserted into the culture tank 20. In this aspect, the first gas supply portion 42a to the fourth gas supply portion 42d and the first guide portion 44a to the fourth guide portion 44d are insertable into and removable from the culture tank 20. When the microalgae and the culture solution L are collected from the culture tank 20, the gas supply portion 42 and the guide portion 44 are not required. Therefore, the microalgae and the culture solution L may be collected from the culture tank 20 after the gas supply portion 42 and the guide portion 44 are removed from the culture tank 20. However, it is also possible to recover the microalgae and the culture solution L from the culture tank 20 in a state where the gas supply portion 42 and the guide portion 44 remain inserted into the culture tank 20.

The first guide portion 44a is adjacent to the side portion 32b of the culture tank 20. The second guide portion 44b is adjacent to the first guide portion 44a at a predetermined interval along the extension direction Y. The third guide portion 44c is adjacent to the second guide portion 44b at a predetermined interval along the extension direction Y. The fourth guide portion 44d is adjacent to the third guide portion 44c at a predetermined interval along the extension direction Y. The interval of separation between the first guide portion 44a and the second guide portion 44b, the interval of separation between the second guide portion 44b and the third guide portion 44c, and the interval of separation between the third guide portion 44c and the fourth guide portion 44d are substantially equal to each other. Although four guide portions of the first guide portion 44a to the fourth guide portion 44d are provided in this aspect, the number of guide portions 44 is not limited to four and is appropriately determined according to the volume of the culture tank 20.

Each of the first guide portion 44a to the fourth guide portion 44d has a hook portion (not shown). The hook portion is held by the holding frame 24, whereby the first guide portion 44a to the fourth guide portion 44d are positioned at predetermined positions in the culture tank 20.

When the first guide portion 44a to the fourth guide portion 44d are viewed from the side portion 32a of the culture tank 20, the first guide portion 44a to the fourth guide portion 44d are hollow bodies having a substantially inverted L-shape. In each of the first guide portion 44a to the fourth guide portion 44d, the lower end and a side portion facing the side portion 32d of the culture tank 20 are open. By this opening, an intake port 46 is formed at each of the first guide portion 44a to the fourth guide portion 44d. That is, all the intake ports 46 of the first guide portion 44a to the fourth guide portion 44d are oriented in the same direction.

The first gas supply portion 42a to the fourth gas supply portion 42d are supported by the first guide portion 44a to fourth guide portion 44d, respectively. One ends of the first gas supply portion 42a to the fourth gas supply portion 42d are connected to the gas supply source 40 shown in FIG. 1. The other ends of the first gas supply portion 42a to the fourth gas supply portion 42d go around to reach the lower openings of the intake ports 46 of the first guide portion 44a to the fourth guide portion 44d. The gas discharge ports 48 are formed at the other ends of the first gas supply portion 42a to the fourth gas supply portion 42d, respectively.

The gas supplied from the gas supply source 40 is discharged from the gas discharge port 48. The gas discharged from the gas discharge port 48 flows into the first guide portion 44a to the fourth guide portion 44d via the lower openings of the intake ports 46 of the first guide portion 44a to the fourth guide portion 44d.

The interior of the water storage tank 22 is a space for storing temperature-regulating water C. The temperature-regulating water C cools the culture solution L in the culture tank 20.

The collection device 14 has a collection tube 50 and a suction device 51. In this aspect, the suction device 51 is a bidirectional pump 52. The bidirectional pump 52 is connected to the collection tank 58 through the first liquid feeding tube 54 and the second liquid feeding tube 56. The bidirectional pump 52 is connected to the collection tube 50 via a supply/discharge tube 60.

The bidirectional pump 52 sends out the culture solution L in the culture tank 20 to the collection tank 58 via the collection tube 50, the supply/discharge tube 60, and the first liquid feeding tube 54. The bidirectional pump 52 also returns the culture solution L in the collection tank 58 to the culture tank 20 via the second liquid feeding tube 56, the supply/discharge tube 60, and the collection tube 50. As can be understood from this structure, the bidirectional pump 52 serves as both a suction device for suctioning the culture solution L in the culture tank 20 and a supply device for sending the culture solution L to the culture tank 20. The collection tube 50 is a tube for collecting the culture solution L from the culture tank 20 and a tube for supplying the culture solution L to the culture tank 20.

The collection tube 50 is described. As shown in FIG. 1, the collection tube 50 has a bottom side collection portion 62 and a relay portion 64. The collection tube 50 is a hollow body with an internal space 68. That is, the collection tube 50 is a tubular body.

The bottom side collection portion 62 is adjacent to the bottom portion 30 of the culture tank 20 below the lower ends of the first gas supply portion 42a to the fourth gas supply portion 42d. The bottom side collection portion 62 extends, in this position, along the horizontal extension direction Y of the culture tank 20. That is, the extending direction of the bottom side collection portion 62 is the same direction as the extending direction Y of the culture tank 20.

The bottom side collection portion 62 has a proximal end portion 66 that is continuous with the lower end of the relay portion 64 and a distal end portion 70 that faces the side portion 32b. The proximal end portion 66 is adjacent to the side portion 32d. The distal end portion 70 is preferably located below the first guide portion 44a. In this aspect, as shown in FIG. 1, the bottom side collection portion 62 extends from one end portion (side portion 32d) of the culture tank 20 in the extension direction Y below the first guide portion 44a. The position of the distal end portion 70 is not particularly limited to this example. The position of the distal end portion 70 may be between the first guide portion 44a and the second guide portion 44b, for example. Preferably, the bottom side collection portion 62 extends from one end portion (side portion 32d) in the extension direction Y of the culture tank 20 to at least an area below the second guide portion 44b.

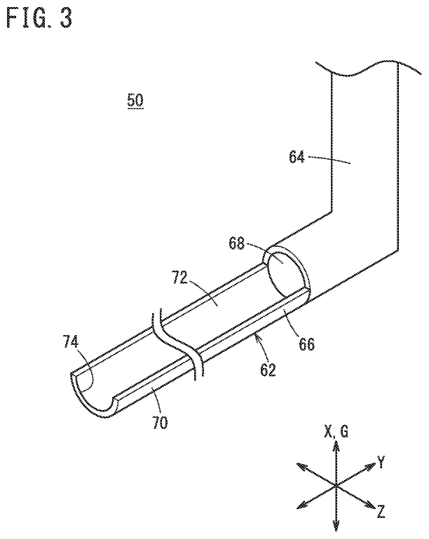

As shown in FIG. 3, the bottom side collection portion 62 is formed with an open port 72 at a portion facing toward the opening 34. The open port 72 extends along the extending direction of the bottom side collection portion 62. Therefore, the cross-section of the bottom side collection portion 62 when viewed along the width direction Z is, for example, semicircular. The internal space 68 of the bottom side collection portion 62 and the interior of the culture tank 20 communicate with each other via the open port 72.

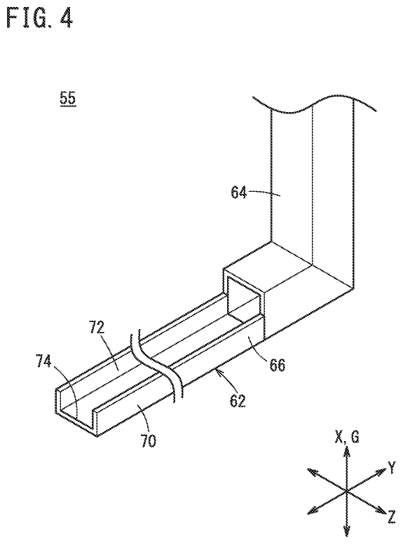

The cross section of the bottom side collection portion 62 when viewed along the width direction z is not particularly limited to a semicircular shape. The cross section of the bottom side collection portion 62 as viewed along the width direction Z may be a quadrangular shape with an open top, as the collection tube 55 shown in FIG. 4, for example. As can be understood from this example, the cross section may be polygonal with an open top. In FIG. 4, the same reference numerals are assigned to the same components as those shown in FIG. 3 for ease of understanding.

As shown in FIGS. 3 and 4, the distal end portion 70 is an open end. That is, a terminal hole 74 is formed at the distal end portion 70. The terminal hole 74 also connects the internal space 68 of the bottom side collection portion 62 with the interior of the culture tank 20. The open port 72 and the terminal hole 74 are continuous with each other.

FIGS. 3 and 4 illustrate the collection tubes 50 and 55, respectively, in which the bottom side collection portion 62 and the relay portion 64 are separate members. In the relay portion 64, the portion bent in an L-shape may also be a separate member. The collection tubes 50, 55 may be single members in which the bottom side collection portion 62 and the relay portion 64 are provided integrally.

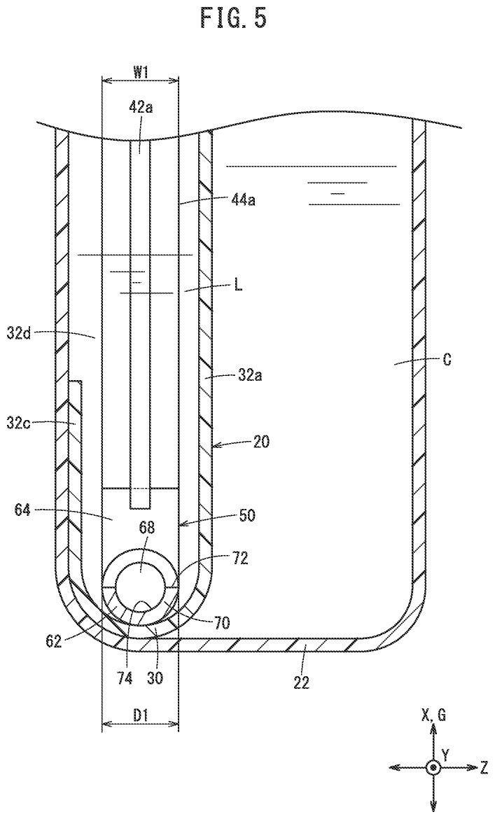

FIG. 5 is an enlarged view of the bottom portion 30 of the culture tank 20 and the bottom portion of the water storage tank 22. The viewing direction in FIG. 5 is the direction from the side portion 32b to the side portion 32d. The bottom side collection portion 62 has a dimension D1 along the width direction Z. The dimension D1 is equal to the outer diameter of bottom side collection portion 62. Therefore, hereinafter the dimension D1 will be referred to as the outer diameter D1. The first guide portion 44a to the fourth guide portion 44d have a width direction dimension W1 along the width direction Z. In this aspect, the outer diameter D1 of the bottom side collection portion 62 and the width direction dimension W1 of the first guide portion 44a to the fourth guide portion 44d are approximately equal to each other. However, the outer diameter D1 and the width direction dimension W1 may be different from each other.

As shown in FIG. 1, the relay portion 64 is continuous with the proximal end portion 66 of the bottom side collection portion 62 in the vicinity of the side portion 32d at the bottom portion 30. The relay portion 64 is continuous with the bottom side collection portion 62 in a manner so that the relay portion 64 bends by approximately 90° with respect to the bottom side collection portion 62. The relay portion 64 further extends along the depth direction X (gravity direction G) of the culture tank 20, adjacent to the side portion 32d of the culture tank 20. Thus, the collection tube 50 has a generally L-shape. The bidirectional pump 52 (suction device 51) is connected to the relay portion 64 via the supply/discharge tube 60.

The relay portion 64 is typically cylindrical in shape. Alternatively, the relay portion 64 may be quadrangular tubular shaped. Although not particularly shown, the relay portion 64 may be formed with suction holes.

The collection tube 50 is inserted into the culture tank 20, for example, in a manner that allows insertion and removal. The collection tube 50 is operated when microalgae and the culture solution L are recovered from the culture tank 20, and is not operated when microalgae are cultured in the culture tank 20. However, when microalgae are cultured in the culture tank 20, it is not particularly necessary to keep the collection tube 50 out of the culture solution L.

The culture system 10 according to the present embodiment is basically configured as described above. Next, the operation of the culture system 10 will be described.

When microalgae are cultured, the culture tank 20 houses the first guide portion 44a to the fourth guide portion 44d, the first gas supply portion 42a to the fourth gas supply portion 42d, the culture solution L, and microalgae. In this embodiment, at this point, the collection tube 50 is also housed in the culture tank 20.

Then, the culture solution L stored in the collection tank 58 is supplied to the culture tank 20 via the collection tube 50. In this case, the bidirectional pump 52 is activated. Thereby, the culture solution L in the collection tank 58 flows through the second liquid feeding tube 56, the supply/discharge tube 60, and the collection tube 50. As described above, the bottom side collection portion 62, which is part of the collection tube 50, is formed with the open port 72 and the terminal hole 74. Therefore, the culture solution L that has reached the internal space 68 of the collection tube 50 is discharged into the culture tank 20 via the open port 72 and the terminal hole 74. When the distal end portion 70 is positioned at least below the second guide portion 44b, the culture solution L can be supplied into the culture tank 20 substantially uniformly.

Then, the gas containing carbon dioxide is supplied from the gas supply source 40 (see FIG. 1). The gas flows through the first gas supply portion 42a to the fourth gas supply portion 42d and then is discharged from the gas discharge port 48. The gas forms bubbles 100 near the lower opening of the intake port 46 of the first guide portion 44a to the fourth guide portion 44d. Here, the intake port 46 of each of the first guide portion 44a to the fourth guide portion 44d is also open at a portion facing the side portion 32d of the culture tank 20. Therefore, when the bubbles 100 rise in the culture solution L while being guided by the first guide portion 44a to the fourth guide portion 44d, the culture solution L around the bubbles 100 is taken in from the side opening of the intake port 46 as indicated by arrows S in FIG. 1. Thereby, the culture solution L around the bubbles 100 is entrained (carried along) by the bubbles 100.

The culture solution L entrained by the bubbles 100 rises along the first guide portion 44a to the fourth guide portion 44d. Thereafter, the traveling direction of the culture solution L and the bubbles 100 are changed by contacting a ceiling portion of each of the first guide portion 44a to the fourth guide portion 44d. That is, the culture solution L and the bubbles 100 that have risen along the first guide portion 44a to the third guide portion 44c flow toward the second guide portion 44b to the fourth guide portion 44d, respectively. The culture solution L and the bubbles 100 that have risen along the fourth guide portion 44d flow toward the side portion 32d of the culture tank 20.

The culture solution L having touched the second guide portion 44b to the fourth guide portion 44d descends along the second guide portion 44b to the fourth guide portion 44d. The culture solution L having touched the side portion 32d of the culture tank 20 descends along the side portion 32d.

The descending culture solution L proceeds toward the bottom side collection portion 62. Here, the bottom side collection portion 62 is formed with the open port 72 at a portion facing the opening 34. Therefore, the cross section of the bottom side collection portion 62 viewed from the width direction Z is substantially semi-cylindrical. Therefore, the culture solution L flows into the internal space 68 of the bottom side collection portion 62 via the open port 72 as shown in FIG. 1.

The culture solution L flows slightly in the internal space 68 toward a lower portion of each of the first guide portion 44a to the fourth guide portion 44d. As described above, the culture solution L in the culture tank 20 is taken in through the intake port 46 of each of the first guide portion 44a to the fourth guide portion 44d. The culture solution L in the internal space 68 is merged with the culture solution L in the culture tank 20 and taken in through the intake port 46.

Based on the above phenomenon, a small amount of convection occurs between the first guide portion 44a and the second guide portion 44b as indicated by an arrow A in FIG. 1. Similarly, small amounts of convection also occur between the second guide portion 44b and the third guide portion 44c, and between the third guide portion 44c and the fourth guide portion 44d, respectively, as indicated by arrows A. A small amount of convection also occurs between the fourth guide portion 44d and the side portion 32d of the culture tank 20, as indicated by an arrow A.

Four of the small amounts of convection combine to generate a large amount of convection in the culture solution L in the culture tank 20 as indicated by an arrow B. The small amounts of convection and the large amount of convection cause the diffusion of the bubbles 100. That is, the carbon dioxide contained in the gas diffuses throughout the culture solution L and is sufficiently dissolved in the culture solution L. Further, the microalgae are agitated by the small amounts of convection and the large amount of convection. As a result, settling or coalescing of microalgae is prevented.

The culture solution L in the culture tank 20 is cooled by the temperature-regulating water C stored in the water storage tank 22. Therefore, the temperature of the culture solution L is prevented from excessively increasing.

For the reasons described above, microalgae are favorably cultured in the entire culture tank 20. Microalgae are exposed to light that is shone at the culture tank 20 during the culturing. As a result, carbon dioxide is sufficiently consumed based on active photosynthesis of microalgae. The light is sunlight or artificial light.

In this way, even when the bottom side collection portion 62 is present in the culture tank 20, the culture solution L can be made to flow by convection based on the formation of the open port 72 at the bottom side collection portion 62. That is, according to the present embodiment, it is possible to avoid a case where the convection of the culture solution L is inhibited by the collection tube 50. Therefore, microalgae can be cultured in a state where the collection tube 50 remains inserted into the culture tank 20.

After microalgae culturing ends, the opening 34 of the culture tank 20 is closed and the bidirectional pump 52 is activated. As a result, the culture solution L and microalgae in the culture tank 20 are drawn into the internal space 68 of the collection tube 50. Specifically, the culture solution L and the microalgae flow into the internal space 68 of the collection tube 50 from the open port 72 and the terminal hole 74 formed for the collection tube 50. The culture solution L and the microalgae are further sent to the collection tank 58 via the supply/discharge tube 60 and the first liquid feeding tube 54.

As the culture solution L and microalgae are drawn out from the culture tank 20, the pressure (internal pressure) of the culture solution in the culture tank 20 decreases. Since the material of the culture tank 20 is flexible, the culture tank 20 with a reduced internal pressure contracts due to the external atmospheric pressure.

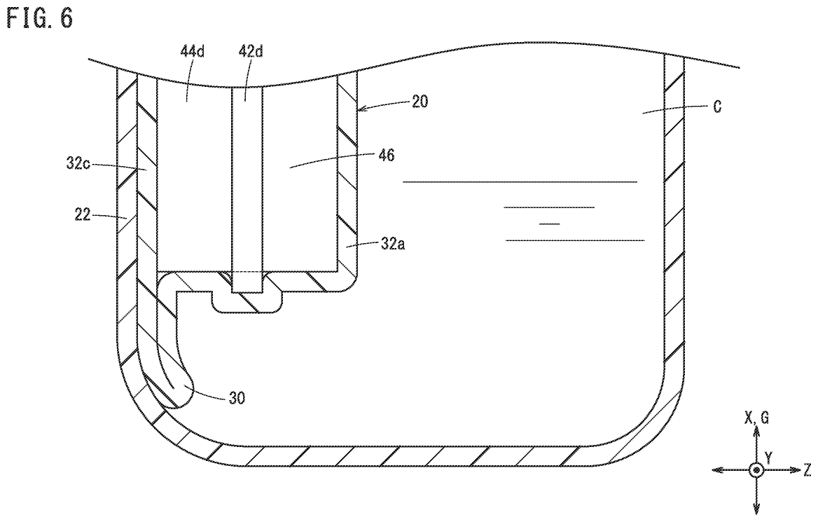

When the collection tube 50 does not have the bottom side collection portion 62, as shown in FIG. 6 where the side portion 32d is seen from the side portion 32b, in the culture tank 20, for example, there is a possibility that a portion below the fourth guide portion 44d may be narrowed by the pressure of the temperature-regulating water C in the water storage tank 22. It is difficult for the culture solution L and microalgae to flow through such a narrowed site. Therefore, it is also difficult to collect the culture solution L of the culture tank 20.

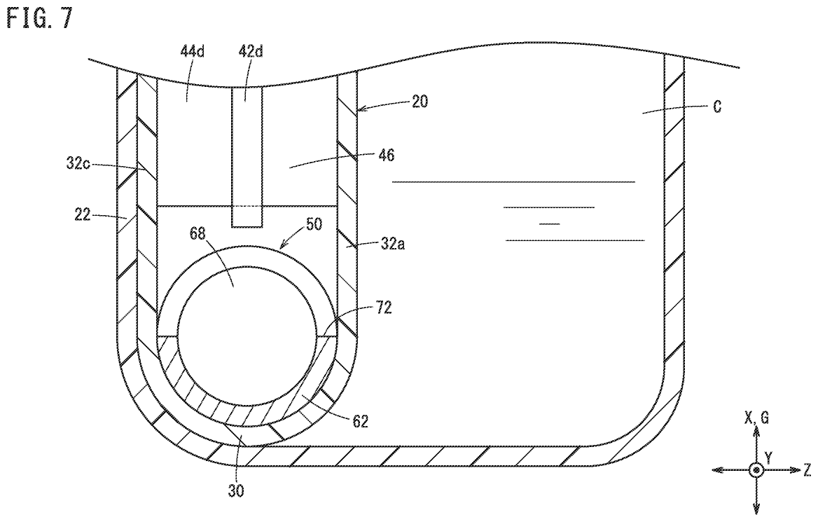

In contrast, in the present embodiment, the collection tube 50 has a bottom side collection portion 62. The outer diameter D1 (width direction dimension) of the bottom side collection portion 62 is approximately equal to the width direction dimension W1 of the first guide portion 44a to the fourth guide portion 44d. Therefore, when the culture tank 20 with a reduced internal pressure contracts due to the external pressure, as shown in FIG. 7 where the side portion 32d is seen from the side portion 32b, the bottom side collection portion 62 and the first guide portion 44a to the fourth guide portion 44d function as stoppers between the side portions 32a and 32c. In other words, the bottom side collection portion 62 and the first guide portion 44a to the fourth guide portion 44d serve as supports that suppress excessive shrinkage of the culture tank 20 between the side portion 32a and the side portion 32c. The support separates the side portions 32a and 32c at a predetermined interval while the culture solution L and microalgae are collected. Therefore, a sufficient amount of the culture solution L and the microalgae can move toward the bottom side collection portion 62. For the reasons described above, the culture solution L and the microalgae can be smoothly guided to the internal space 68 of the bottom side collection portion 62 via the open port 72 and the terminal hole 74.

The bottom side collection portion 62 extends from the side portion 32b at least up to an area below the second guide portion 44b. The bottom side collection portion 62 preferably extends from the side portion 32b up to an area below the first guide portion 44a. Therefore, the culture solution L and the microalgae between the first guide portion 44a and the second guide portion 44b can be sufficiently collected by the bottom side collection portion 62, despite the existence of the third guide portion 44c and the fourth guide portion 44d in the culture solution L. That is, the culture solution L and the microalgae can be recovered almost uniformly over the entire inside of the culture tank 20.

As can be understood from the above, according to the present embodiment, it is possible to extract approximately the entire amount of the culture solution L and the microalgae from the culture tank 20 while suppressing the formation of wrinkles in the culture tank 20.

As the culture tank 20 contracts, the side portions 32a, 32b, and 32c of the culture tank 20 are pulled toward the bottom side collection portion 62. In this embodiment, the distal end portion 70 of the bottom side collection portion 62 is located below the first guide portion 44a. That is, a clearance is formed between the side portion 32b and the distal end portion 70. Therefore, when the culture tank 20 is contracted, the distal end portion 70 is avoided from contacting the side portion 32b. Therefore, the distal end portion 70 is prevented from penetrating the culture tank 20.

Meanwhile, it is assumed that the side portions 32a and 32c come into contact with the outer surface of the bottom side collection portion 62. Since the bottom side collection portion 62 has a substantially semi-cylindrical shape, there are no corners on the outer surface of the bottom side collection portion 62. Therefore, even when the side portions 32a and 32c contact the outer surface of the bottom side collection portion 62, the bottom side collection portion 62 is prevented from penetrating through the side portions 32a and 32c.

It is also expected that the side portions 32a, 32b, and 32d of the culture tank 20 will come into contact with the outer surface of the relay portion 64 as the culture tank 20 contracts. Here, since the relay portion 64 has a cylindrical shape, there are no corner portions on the outer surface of the relay portion 64. Therefore, even when the side portions 32a, 32b, and 32d contact the relay portion 64, the relay portion 64 is prevented from penetrating the side portions 32a, 32b, and 32d.

The microalgae collected in the collection tank 58 are separated from the culture solution L by a known method such as centrifugation, coagulation separation, or spontaneous sedimentation. The culture solution L remaining in the collection tank 58 is then drawn out from the bottom portion of the collection tank 58 and returned to the culture tank 20 via the second liquid feeding tube 56, the supply/discharge tube 60, and the collection tube 50. The culture solution L is preferably returned to the culture tank 20 after being subjected to component adjustment so as to be suitable for culturing microalgae. Thereafter, microalgae cultivation is performed in the same manner as described above.

By allowing the culture solution L to be supplied into the culture tank 20 via the collection tube 50 in this manner, it is unnecessary to provide a supply tube separate from the collection tube 50 in the culture system 10. Therefore, the configuration of the culture system 10 is simplified. In addition, the same culture solution L can be used repeatedly for culturing, which can save resources.

Moreover, a space is formed below the first guide portion 44a to the fourth guide portion 44d by the bottom side collection portion 62. Therefore, the culture solution L can be uniformly supplied to the entire culture tank 20.

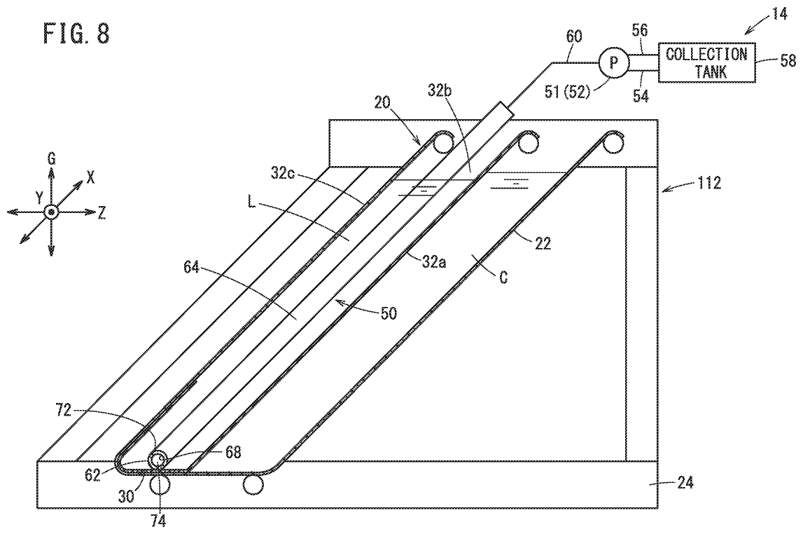

FIGS. 1-7 show aspects in which the depth direction X of the culture tank 20 approximately matches the gravity direction G. In contrast, in the case of a culture device 112 shown in FIG. 8, the depth direction X of the culture tank 20 is inclined with respect to the gravity direction G. Also in this aspect, the same effects as those in the aspects shown in FIGS. 1 to 7 are acquired.

As described above, the present embodiment discloses a culture system (10) including a culture device (12) that includes a culture tank (20) for culturing microalgae, and a collection device (14) that collects the microalgae from the culture tank, wherein the culture tank contains a culture solution (L) and includes a bottom portion (30) located at the lowest position in a depth direction (X) and an opening (34) located at a position higher than the bottom portion, the culture device includes a gas supply source (40) and a gas supply portion (42) inserted into the culture tank through the opening portion, the gas supply portion includes a gas discharge port (48) that opens in the culture solution at a lower position in the depth direction and is configured to discharge the gas supplied from the gas supply source, the collection device includes a collection tube (50) housed in the culture tank and a suction device (51), the collection tube is a tubular body having an internal space (68), the collection tube includes a bottom side collection portion (62) that extends along the bottom portion of the culture tank when immersed in the culture solution (L) in the culture tank and is positioned below the gas supply portion in the depth direction, and, and the bottom side collection portion includes an open port (72) formed at a site facing the opening.

The gas discharged into the culture solution from the lower part of the culture tank in the depth direction rises toward the upper part of the culture tank in the depth direction based on the specific gravity difference with the culture solution. Based on this gas flow, convection occurs in the culture solution L in the culture tank. That is, the microalgae and the culture solution flow. Specifically, the microalgae and the culture solution rise with the gas and then fall. This causes agitation of the microalgae and the culture solution.

Here, the bottom side collection portion, which is part of the collection tube, has an open port that opens at a site facing the opening of the culture tank. When the stream of microalgae and culture solution reaches the bottom side collection portion, the stream flows into the internal space of the bottom side collection portion from the open port and then returns to the culture tank from the internal space of the bottom side collection through the open port.

As can be understood from the above, the flow of microalgae and culture solution is not inhibited by the bottom side collection portion by forming the open port at the bottom side collection portion. Therefore, microalgae and culture solution are sufficiently agitated. This enhances microalgae cultivation.

Moreover, for the reasons described above, microalgae can be cultured in a state where the collection tube is accommodated in the culture tank. Therefore, it is not particularly necessary to remove the collection tube from the culture tank each time microalgae are cultured. Hence, no cumbersome work is required.

The present embodiment discloses a culture system wherein the culture device includes a guide portion (44) that is inserted into the culture tank from the opening and extends in the depth direction, and guides the gas discharged from the gas discharge port, and the bottom side collection portion is located below the guide portion in the depth direction.

In this configuration, the guide portion defines the direction of the flow of gas and the direction of the flow of culture solution and microalgae. In addition, since the bottom side collection portion is located below the guide portion in the depth direction, the rise of the gas in the culture tank is not hindered by the bottom side collection portion.

This embodiment discloses a culture system wherein the culture device includes a water storage tank (22) that contains temperature-regulating water (C) for regulating the temperature of the culture solution and is adjacent to the culture tank, and the culture tank is made of a material that exhibits flexibility.

If the culture device has a water storage tank, when the microalgae and the culture solution in the culture tank are collected (suctioned) through the collection portion that does not have a bottom side collection portion, there is a concern that part of the culture tank will be crushed by the pressure of the temperature-regulating water in the water storage tank. In other words, the flow path of the culture solution in the culture solution is significantly narrowed. It is not easy for culture solution to pass through a narrow path. Therefore, in this case, it is not easy to collect the culture solution and microalgae.

In contrast, according to the above configuration, the bottom side collection portion serves as a support for suppressing excessive shrinkage of the culture tank. That is, the bottom side collection portion functions as a stopper for the culture tank. In this way, the bottom side collection portion supports the inner surface of the culture tank to prevent the culture tank from collapsing. This ensures a flow path for the culture solution in the culture solution. Therefore, it is possible to easily recover the culture solution and microalgae.

This embodiment discloses a culture system in which the dimension (D1) of the bottom side collection portion in a width direction (Z) is approximately equal to the dimension (W1) of the guide portion in the width direction when the width direction is a direction parallel to a horizontal direction and is orthogonal to an extending direction of the bottom side collection portion.

In this case, the guide portion functions as a stopper for the culture tank. That is, the guide portion supports the inner surface of the culture tank to prevent the culture tank from collapsing. As a result, a sufficient flow path for the culture solution is ensured in the culture solution. Therefore, the culture solution and microalgae can be collected more easily.

The present embodiment discloses a culture system, wherein the collection tube includes a relay portion (64) at one end portion in an extending direction of the culture tank, the relay portion connecting a one end portion in an extending direction of the bottom side collection portion and the suction device with each other, and extending in the depth direction, the relay portion is formed in a cylindrical shape, and the bottom side collection portion is formed in a semicircular shape in cross section by the open port.

Since the bottom side collection portion has a semicircular cross section, there are no corners on the outer surface of the bottom side collection portion. Therefore, when the culture solution is collected (suctioned) from the culture tank, even if the contracted culture tank contacts the bottom side collection portion, the bottom side collection portion is prevented from penetrating the culture tank.

This embodiment discloses a culture system, wherein the collection tube includes a relay portion (64) at one end portion in an extending direction along a horizontal direction in the culture tank, the relay portion connecting a one end portion in an extending direction of the bottom side collection portion and the suction device with each other, and extending in the depth direction, the guide portion includes a first guide portion (44a) and a second guide portion (44b) that are arranged with an interval in between in the extending direction of the culture tank, the first guide portion is adjacent to another end portion in the extending direction of the culture tank and extends in the depth direction, the second guide portion is positioned between the first guide portion and the relay portion in the extending direction of the culture tank, and the bottom side collection portion extends from the one end portion in the extending direction of the culture tank up to at least an area below the second guide portion.

The bottom side collection portion extends from one end portion in the extending direction of the culture tank to an area between the first guide portion and the second guide portion. Therefore, even when another guide portion is provided between the second guide portion and the relay portion, it is easy to collect the culture solution in the culture tank substantially uniformly.

This embodiment discloses a culture system wherein the bottom side collection portion extends from the one end portion in the extending direction of the culture tank up to an area below the first guide portion.

In this case, the distal end portion of the bottom side collection portion is located below the first guide portion. Therefore, when the culture tank is contracted, the distal end portion of the bottom side collection portion is prevented from contacting another end portion of the culture tank in the extending direction. Therefore, the distal end portion of the bottom side collection portion is prevented from penetrating the culture tank.

This embodiment discloses a culture system configured to supply the culture solution into the culture tank through the collection tube.

In this case, the collection tube also serves as a supply tube for supplying the culture solution. Therefore, it is not necessary to provide the collection tube and the supply tube separately in the culture tank. Therefore, the culture system is simplified. In some cases, the same culture solution can be used repeatedly for cultivation. Therefore, resource saving can be achieved. Moreover, in the culture tank, sufficient space is formed below the guide portion by the bottom side collection portion. Therefore, the culture solution can be uniformly supplied to the entire culture tank.

The present invention is not limited to the above disclosure, and various configurations can be employed without departing from the gist of the present invention.

What is claimed is:

1. A culture system comprising a culture device that includes a culture tank for culturing microalgae, and a collection device that collects the microalgae from the culture tank, wherein
the culture tank contains a culture solution and includes a bottom portion located at a lowest position in a depth direction and an opening located at a position higher than the bottom portion,
the culture device includes a gas supply source and a gas supply portion inserted into the culture tank through the opening,
the gas supply portion includes a gas discharge port that opens in the culture solution at a lower position in the depth direction and is configured to discharge gas supplied from the gas supply source,
the collection device includes a collection tube housed in the culture tank and a suction device,
the collection tube is a tubular body having an internal space,
the collection tube includes a bottom side collection portion that extends along the bottom portion of the culture tank when immersed in the culture solution in the culture tank and is positioned below the gas supply portion in the depth direction, and
the bottom side collection portion includes an open port formed at a site facing the opening.

2. The culture system according to claim 1, wherein the culture device includes a guide portion that is inserted into the culture tank from the opening and extends in the depth direction, and guides the gas discharged from the gas discharge port, and
the bottom side collection portion is located below the guide portion in the depth direction.

3. The culture system according to claim 2, wherein the culture device includes a water storage tank that contains temperature-regulating water for regulating a temperature of the culture solution and is adjacent to the culture tank, and
the culture tank is made of a material that exhibits flexibility.

4. The culture system according to claim 3, wherein a dimension of the bottom side collection portion in a width direction is approximately equal to a dimension of the guide portion in the width direction when the width direction is a direction parallel to a horizontal direction and is orthogonal to an extending direction of the bottom side collection portion.

5. The culture system according to claim 3, wherein the collection tube includes a relay portion at one end portion in an extending direction of the culture tank, the relay portion connecting a one end portion in an extending direction of the bottom side collection portion and the suction device with each other, and extending in the depth direction,
the relay portion is formed in a cylindrical shape, and
the bottom side collection portion is formed in a semicircular shape in cross section by the open port.

6. The culture system according to claim 3, wherein the collection tube includes a relay portion at one end portion in an extending direction along a horizontal direction in the culture tank, the relay portion connecting a one end portion in an extending direction of the bottom side collection portion and the suction device with each other, and extending in the depth direction,
the guide portion includes a first guide portion and a second guide portion that are arranged with an interval in between in the extending direction of the culture tank,
the first guide portion is adjacent to another end portion in the extending direction of the culture tank and extends in the depth direction,
the second guide portion is positioned between the first guide portion and the relay portion in the extending direction of the culture tank, and
the bottom side collection portion extends from the one end portion in the extending direction of the culture tank up to at least an area below the second guide portion.

7. The culture system according to claim 6, wherein the bottom side collection portion extends from the one end portion in the extending direction of the culture tank up to an area below the first guide portion.

8. The culture system according to claim 1, wherein the culture system is configured to supply the culture solution into the culture tank through the collection tube.
